(19) United States
(12) Patent Application Publication
(10) Pub. No.: US 2019/0343441 A1
(43) Pub. Date: Nov. 14, 2019

(54) COGNITIVE DIVERSION OF A CHILD DURING MEDICAL TREATMENT

(71) Applicant: International Business Machines Corporation, Armonk, NY (US)

(72) Inventors: Sarbajit K. Rakshit, Kolkata (IN); Craig Trim, Ventura, CA (US); Victor Povar, Vancouver (CA); Martin G. Keen, Cary, NC (US)

(21) Appl. No.: 15/975,756

(22) Filed: May 9, 2018

(51) Int. Cl. A61B 5/16; G16H 40/63; G09B 19/00; A61B 5/1171; A61B 5/0205

(52) U.S. Cl. CPC A61B 5/165; G16H 40/63; A61B 5/024; A61B 5/1176; A61B 5/02055; G09B 19/00

(57) ABSTRACT

The cognitive diversion of a child patient during medical treatment includes receiving an image of a child and processing the image to determine a contemporaneous emotional state, selecting a specific procedure and retrieving a requisite minimum state of distraction necessary for the procedure and comparing the contemporaneous emotional state to the requisite minimum state of distraction. On condition that the contemporaneous emotional state lacks the requisite minimum state of distraction, an activity is identified in a table that correlates to a degree of diversion exceeding the requisite minimum state of distraction, the identified activity is presented in a display and additional imagery of the child processed while the child engages in the identified activity in order to re-determine the contemporaneous emotional state. Once the contemporaneous emotional state exceeds the requisite minimum state of distraction, an alert is generated indicating a readiness of the child patient to receive the selected procedure.

### BACKGROUND OF THE INVENTION

### Field of the Invention

The present invention relates to the field of health care delivery and more particularly to the medical treatment of a child.

### Description of the Related Art

Convention health care includes as a foundational aspect, the office visit. During an office visit, the patient interacts directly with the health care provider—typically a physician or nurse. The health care practitioner-patient interaction generally includes physical contact between the practitioner and patient, utilizing the hands of the practitioner, as well as various tools of the trade. Of course, oftentimes, a needle is present during the encounter so as to deliver an injection to the patient or to draw blood from the patient. The traditional health care encounter also may include one or more imaging processes, such as the use of an x-ray, magnetic resonance imaging device, computer aided tomography scanner or ultrasound wand.

For the adult patient, an ordinary health care encounter may range from the routine to the anxiety provoking. But, for most, as an adult with the perspective of many years of experience, the encounter remains tolerable such that the health care practitioner may effectively conduct a medical evaluation or treatment with the full cooperation of the patient. So much cannot be said of the child patient, though. The child patient lacks the experience of the adult patient and may be easily intimidated by any number of aspects of the typical health care encounter, not the least of which includes the administration of a vaccine or the drawing of blood by way of a hypodermic needle. Indeed, the degree of anxiety expressed by a child patient under such circumstances often translates to non-cooperative behavior inhibiting the health care provider from effectively conducting a medical evaluation or treatment of the child patient. In extreme circumstances, needed treatment can become so inhibited as to require the use of measures such as the imposition of restraints upon the child or the administration of sedatives to the child.

### BRIEF SUMMARY OF THE INVENTION

Embodiments of the present invention address deficiencies of the art in respect to the delivery of treatment to a child patient during a health care encounter and provide a novel and non-obvious method, system and computer program product for the cognitive diversion of a child patient during medical treatment. In an embodiment of the invention, a method for cognitive diversion of a child patient during medical treatment includes first receiving in memory of a computer data processing system, during a health care practitioner encounter involving a child patient, either or both of an image of the child patient and also sensed biometric data of the child patient as provided from a wearable device coupled to the child patient. Either or both of the image and the biometric data are then processed to determine a contemporaneous emotional state. As well, a specific medical procedure is selected and a requisite minimum state of distraction necessary for the selected specific medical procedure determined.

Thereafter, the contemporaneous emotional state may be compared to the requisite minimum state of distraction. As such, on the condition that the contemporaneous emotional state is determined in the comparison to lack the requisite minimum state of distraction, an activity may be identified in a table that is correlated with a degree of diversion that exceeds the requisite minimum state of distraction. Then, the identified activity is presented in a display of the computer data processing system, for example the playback in the computer data processing system of particular media content. While the child patient engages in the identified activity, additional imagery of the child patient is processed in order to re-determine the contemporaneous emotional state. Finally, subsequent to the contemporaneous emotional state exceeding the requisite minimum state of distraction, an alert is generated in the computer data processing system, for instance a text message directed for transmission to a mobile device of a health care practitioner assigned to the health care practitioner encounter, indicating a readiness of the child patient to receive the selected specific medical procedure.

In one aspect of the embodiment, the table itself may be generated through the receipt in memory of the computer data processing system from over a computer communications network, of biometric data pertaining to different children in different geographic locations, and also diversion data for each of the different children engaging in different activities. Then, the biometric data is computationally correlated with the diversion data and the different activities in a table in the memory so as to record a cause-and-effect relationship between each one of the activities and a degree of diversion observed in the different children. Optionally, the table includes different manually supplied entries correlating a degree of diversion observed in respectively different ones of the different children and corresponding ones of the different activities. Thereafter, the table is stored in fixed storage of the computer data processing system.

In another embodiment of the invention, a computer data processing system may be adapted for cognitive diversion of a child patient during medical treatment. The system includes a host computing system including one or more computers, each with memory and at least one processor. At least one of the computers is positioned in a treatment room of a health care facility and includes a display and an imaging device. The system further includes fixed storage coupled to the host computing system and a table stored in the fixed storage correlating different activities with different degrees of diversion. Finally, the system includes a cognitive diversion module.

The module includes program instructions executing in the memory of the host computing system, which instructions perform receiving in the memory from the imaging device an image of a child patient during a health care practitioner encounter in the treatment room and processing the image to determine a contemporaneous emotional state of the child patient, selecting in the memory a specific medical procedure and retrieving from the memory, a requisite minimum state of distraction necessary for the selected specific medical procedure, comparing the contemporaneous emotional state to the requisite minimum state of distraction, and on condition that the contemporaneous emotional state is determined in the comparison to lack the requisite minimum state of distraction, identifying in the table an activity correlated with a degree of diversion that exceeds the requisite minimum state of distraction, presenting the identified activity in a display of the computer data processing system, processing additional imagery of the child patient while the child patient engages in the identified activity in order to re-determine the contemporaneous emotional state and, subsequent to the contemporaneous emotional state exceeding the requisite minimum state of distraction, generating an alert indicating a readiness of the child patient to receive the selected specific medical procedure.

Additional aspects of the invention will be set forth in part in the description which follows, and in part will be obvious from the description, or may be learned by practice of the invention. The aspects of the invention will be realized and attained by means of the elements and combinations particularly pointed out in the appended claims. It is to be understood that both the foregoing general description and the following detailed description are exemplary and explanatory only and are not restrictive of the invention, as claimed.

### BRIEF DESCRIPTION OF THE SEVERAL VIEWS OF THE DRAWINGS

The accompanying drawings, which are incorporated in and constitute part of this specification, illustrate embodiments of the invention and together with the description, serve to explain the principles of the invention. The embodiments illustrated herein are presently preferred, it being understood, however, that the invention is not limited to the precise arrangements and instrumentalities shown, wherein:

FIG. 1 is pictorial illustration of a process for cognitive diversion of a child patient during medical treatment;

FIG. 2 is a schematic illustration of a computer data processing system adapted for the cognitive diversion of a child patient during medical treatment; and,

FIG. 3 is a flow chart illustrating a process for the cognitive diversion of a child patient during medical treatment.

### DETAILED DESCRIPTION OF THE INVENTION

Embodiments of the invention provide for the cognitive diversion of a child patient during medical treatment. In accordance with an embodiment of the invention, a child patient during medical treatment is observed biometrically and with an imaging device. Data retrieved from one or more biometric sensors and the imaging device are processed to compute a contemporaneous emotional state. A lookup is then performed in connection with a proposed medical procedure to determine a requisite degree of distraction for a child patient in order to conduct the proposed medical procedure. The requisite degree of distraction is then compared to the contemporaneous emotional state in order to determine whether one or more distractive measures are required. If so, a set of prospective measures are consulted and one of the prospective measures selected and based upon a pre-determined state change effectiveness of the one of the prospective measures and administered to the child patient. Additional data is then retrieved from the sensors and imaging device in order to compute a new emotional state. To the extent that the new emotional state exceeds the requisite degree of distraction, a message is transmitted to a health care provider that the child patient is now ready to receive the proposed medical procedure. In this way, the child patient is optimally distracted for the proposed medical procedure while permitting the health care provider to proceed in other tasks pending the child patient's readiness to receive the proposed medical procedure.

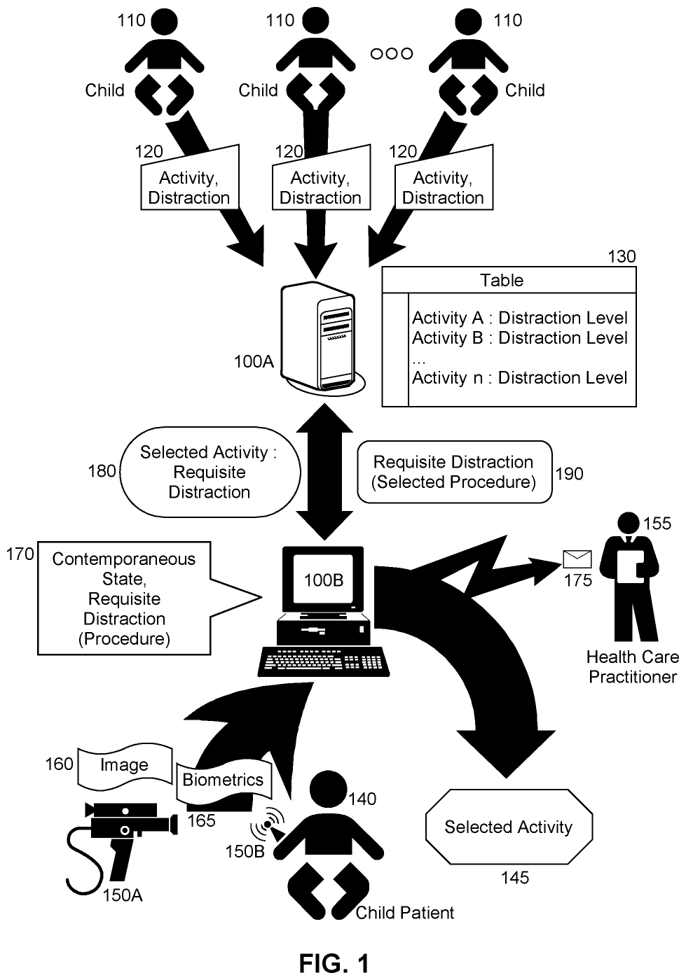

In further illustration, FIG. 1 pictorially shows a process for cognitive diversion of a child patient during medical treatment. As shown in FIG. 1, different children 110 may be monitored for levels of distraction while engaging in different activities such as watching video content, or playing with different toys, or viewing different graphic images. The levels of distraction may be determined biometrically by monitoring the heart rate, temperature or other measurable biometric attribute of each of the children 110 while engaged in the activities, or by viewing facial expressions of the children 110 while engaged in the activities. It is of note that digitally acquired imagery of the children 110 may be processed through content-based image feature matching or utilizing known image processing techniques in artificial intelligence so as to determine a particular emotional state from each processed image. Distraction data 120 pertaining to the children 110 and incorporating pairs of activity identification data and corresponding levels of distraction may then be provided to a computer data processing system 100A which in turn stores the distraction data 120 in a table 130.

Thereafter, a child patient 140 in the setting of a health care encounter may be monitored with an imaging device 150A producing imagery 160 of the child patient 140, or a biometric sensor 150B, such as a wearable device that reports heart rate, temperature and other such biometric measurements 165. Client computer 100B receives the imagery 160 and biometrics 165 and determines a contemporaneous emotional state of the child patient 140 utilizing image processing of the imagery 160 to identify facial expressions, or the mapping of the biometric measurements 165 to known emotional states. A medical procedure is then selected for the child patient 140 and a requisite minimum level of distraction determined for the selected procedure 190. The contemporaneous emotional state of the child patient 140 and the requisite level of distraction 190 are then presented to a comparator 170 in the client computer 100B so as to determine if the child patient 140 is sufficiently distracted so as to not impeded the performance of the selected medical procedure.

On the condition that child patient 140 is determined in the client computer 100B not to be sufficiently distracted, the client computer 100B queries the table 130 to identify an activity in the table 130 correlated to a particular level of distraction necessary to meet or exceed the requisite level of distraction 190. Thereafter, the selected activity 145 is presented to the child patient 140. For instance, the selected activity 145 may include video content such as a film or television programming, a series of images a projected hologram, a virtual reality presentation or a computer driven robotic toy. As the child patient 140 interacts with the selected activity 145, the client computer 100B continues to monitor the emotional state of the child patient 140 by acquiring further imagery 160 and biometric measurements 165. Once the client computer 100B determines that the contemporaneous emotional state of the child patient 140 demonstrates a level of distraction beyond the requisite level of distraction 190, the client computer 100B generates an alert 175 such as a text message or e-mail message to a mobile device of a health care practitioner 155 assigned to perform the selected procedure.

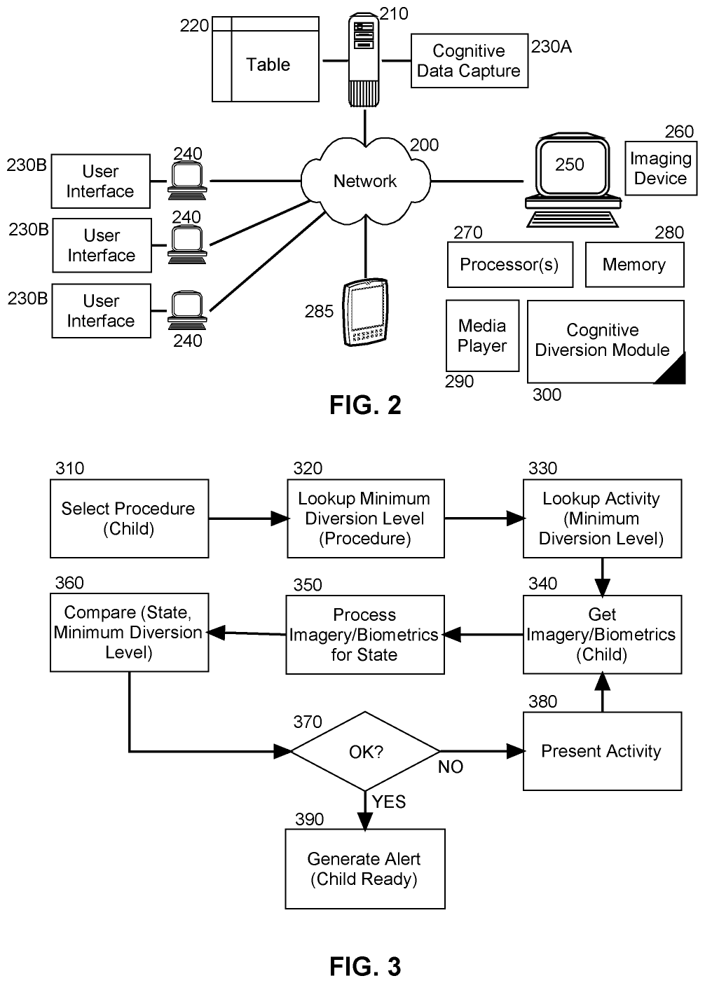

The process described in connection with FIG. 1 may be implemented in a computer data processing system. In further illustration, FIG. 2 schematically shows a computer data processing system adapted for the cognitive diversion of a child patient during medical treatment. The system includes a host server 210 storing therein a table 220 accessible through a cognitive data capture application 230A from over computer communications network 200 by different client devices 240 supporting the presentation therein of a user interface 230B to the cognitive data capture application 230A. In this regard, the cognitive data capture application 230A provides a set of programmatic functions that receive both activity data and corresponding levels of distraction for different children provided through the user interface 230B. The activity data and corresponding levels of distraction may be manually input in the user interface 230B, or computationally acquired by reading biometric data from each of the children, or by processing imagery of the children, in each circumstance as the children engage in a corresponding activity.

Different client computers 250 also are coupled to the host server 210 over the computer communications network 200 and are disposed in different rooms of different health care facilities in which health care encounters occur between children and different health care practitioners. Each of the client computers 250 includes one or more processors 270, memory 280 and an imaging device 260 adapted to acquire imagery of a subject child present in a corresponding room of a corresponding health care facility. Each of the client computers 250 also includes a media player 290 executing in the memory 280 and displaying media content in a display. Even further, each of the client computers 250 includes a cognitive diversion module 300 executing in the memory 280.

The cognitive diversion module 300 includes computer program instructions that when executing in the memory 280, are enabled to receive imagery from the imaging device 260 of a child patient present in the room, or biometric data acquired from the child patient and presented to a corresponding one of the client computers 250 either through short range wireless communications such as Bluetooth™ or through the global Internet. The program instructions are further enabled during execution, to process the imagery and biometric data in order to determine a contemporaneous emotional state of the child patient. The program instructions are yet further enabled during execution to compare the contemporaneous emotional state to a requisite level of distraction for a selected medical procedure to be performed in connection with the child patient.

To the extent that the program instructions determine that the contemporaneous emotional state does not meet or exceed the requisite level of distraction, the program instructions during execution access over the computer communications network 200 the table 220 so as to identify an activity stored in a record therein corresponding to a level of distraction that meets or exceeds the requisite level of distraction. Thereafter, the program instructions during execution are enabled to initiate the identified activity, for instance by playing back in the media player 290 particular media content. As the child patient engages in the identified activity, program instructions that when executing in the memory 280, the program instructions are enabled to continue to receive imagery from the imaging device 260 of the child patient, or the biometric data acquired from the child patient, and to process the imagery and biometric data in order to determine a change in the contemporaneous emotional state of the child patient. To the extent that a change in the contemporaneous emotional state is determined by the program instructions to meet or exceed the requisite level of distraction, the program instructions are enabled to generate an alert to the health care practitioner to begin performance of the selected medical procedure.

In even yet further illustration of the operation of the cognitive diversion module, FIG. 3 is a flow chart illustrating a process for the cognitive diversion of a child patient during medical treatment. Beginning in block 310, a medical procedure is selected for a child patient and in block 320, a minimum level of distraction is identified for the selected procedure. In block 330, an activity is identified in the table that is sufficient to provide for the minimum level of distraction and in block 340, either or both of imagery and biometric data is acquired for the child patient. In block 350, the imagery and/or biometric data is processed to determine a contemporaneous emotional state of the child patient. As such, in block 360 the contemporaneous emotional state is compared to the requisite level of distraction for the selected medical procedure. In decision block 370, if the contemporaneous emotional state falls short of the requisite level of distraction, in block 380 the identified activity is presented to the child patient and the process returns to block 340. However, once the contemporaneous emotional state of the child patient is determined in decision block 370 to meet or exceed the requisite level of distraction, in block 390 and alert is generated indicating the sufficient distraction of the child patient so as to not inhibit performance of the selected medical procedure.

The present invention may be a system, a method, and/or a computer program product at any possible technical detail level of integration. The computer program product may include a computer readable storage medium (or media) having computer readable program instructions thereon for causing a processor to carry out aspects of the present invention.

The computer readable storage medium can be a tangible device that can retain and store instructions for use by an instruction execution device. The computer readable storage medium may be, for example, but is not limited to, an electronic storage device, a magnetic storage device, an optical storage device, an electromagnetic storage device, a semiconductor storage device, or any suitable combination of the foregoing. A non-exhaustive list of more specific examples of the computer readable storage medium includes the following: a portable computer diskette, a hard disk, a random access memory (RAM), a read-only memory (ROM), an erasable programmable read-only memory (EPROM or Flash memory), a static random access memory (SRAM), a portable compact disc read-only memory (CD-ROM), a digital versatile disk (DVD), a memory stick, a floppy disk, a mechanically encoded device such as punch-cards or raised structures in a groove having instructions recorded thereon, and any suitable combination of the foregoing. A computer readable storage medium, as used herein, is not to be construed as being transitory signals per se, such as radio waves or other freely propagating electromagnetic waves, electromagnetic waves propagating through a waveguide or other transmission media (e.g., light pulses passing through a fiber-optic cable), or electrical signals transmitted through a wire.

Computer readable program instructions described herein can be downloaded to respective computing/processing devices from a computer readable storage medium or to an external computer or external storage device via a network, for example, the Internet, a local area network, a wide area network and/or a wireless network. The network may comprise copper transmission cables, optical transmission fibers, wireless transmission, routers, firewalls, switches, gateway computers and/or edge servers. A network adapter card or network interface in each computing/processing device receives computer readable program instructions from the network and forwards the computer readable program instructions for storage in a computer readable storage medium within the respective computing/processing device.

Computer readable program instructions for carrying out operations of the present invention may be assembler instructions, instruction-set-architecture (ISA) instructions, machine instructions, machine dependent instructions, microcode, firmware instructions, state-setting data, configuration data for integrated circuitry, or either source code or object code written in any combination of one or more programming languages, including an object oriented programming language such as Smalltalk, C++, or the like, and procedural programming languages, such as the “C” programming language or similar programming languages. The computer readable program instructions may execute entirely on the user's computer, partly on the user's computer, as a stand-alone software package, partly on the user's computer and partly on a remote computer or entirely on the remote computer or server.

In the latter scenario, the remote computer may be connected to the user's computer through any type of network, including a local area network (LAN) or a wide area network (WAN), or the connection may be made to an external computer (for example, through the Internet using an Internet Service Provider). In some embodiments, electronic circuitry including, for example, programmable logic circuitry, field-programmable gate arrays (FPGA), or programmable logic arrays (PLA) may execute the computer readable program instructions by utilizing state information of the computer readable program instructions to personalize the electronic circuitry, in order to perform aspects of the present invention.

Aspects of the present invention are described herein with reference to flowchart illustrations and/or block diagrams of methods, apparatus (systems), and computer program products according to embodiments of the invention. It will be understood that each block of the flowchart illustrations and/or block diagrams, and combinations of blocks in the flowchart illustrations and/or block diagrams, can be implemented by computer readable program instructions.

These computer readable program instructions may be provided to a processor of a general purpose computer, special purpose computer, or other programmable data processing apparatus to produce a machine, such that the instructions, which execute via the processor of the computer or other programmable data processing apparatus, create means for implementing the functions/acts specified in the flowchart and/or block diagram block or blocks. These computer readable program instructions may also be stored in a computer readable storage medium that can direct a computer, a programmable data processing apparatus, and/or other devices to function in a particular manner, such that the computer readable storage medium having instructions stored therein comprises an article of manufacture including instructions which implement aspects of the function/act specified in the flowchart and/or block diagram block or blocks.

The computer readable program instructions may also be loaded onto a computer, other programmable data processing apparatus, or other device to cause a series of operational steps to be performed on the computer, other programmable apparatus or other device to produce a computer implemented process, such that the instructions which execute on the computer, other programmable apparatus, or other device implement the functions/acts specified in the flowchart and/or block diagram block or blocks.

The flowchart and block diagrams in the Figures illustrate the architecture, functionality, and operation of possible implementations of systems, methods, and computer program products according to various embodiments of the present invention. In this regard, each block in the flowchart or block diagrams may represent a module, segment, or portion of instructions, which comprises one or more executable instructions for implementing the specified logical function(s). In some alternative implementations, the functions noted in the blocks may occur out of the order noted in the Figures. For example, two blocks shown in succession may, in fact, be executed substantially concurrently, or the blocks may sometimes be executed in the reverse order, depending upon the functionality involved. It will also be noted that each block of the block diagrams and/or flowchart illustration, and combinations of blocks in the block diagrams and/or flowchart illustration, can be implemented by special purpose hardware-based systems that perform the specified functions or acts or carry out combinations of special purpose hardware and computer instructions.

We claim:

1. A method for cognitive diversion of a child patient during medical treatment, the method comprising:
receiving in memory of a computer data processing system, an image of a child patient during a health care practitioner encounter and processing the image to determine a contemporaneous emotional state;
selecting in the memory of the computer data processing system a specific medical procedure and retrieving from the memory, a requisite minimum state of distraction necessary for the selected specific medical procedure;
comparing the contemporaneous emotional state to the requisite minimum state of distraction; and,
on condition that the contemporaneous emotional state is determined in the comparison to lack the requisite minimum state of distraction, identifying in a table an activity correlated with a degree of diversion that exceeds the requisite minimum state of distraction, presenting the identified activity in a display of the computer data processing system, processing additional imagery of the child patient while the child patient engages in the identified activity in order to re-determine the contemporaneous emotional state and, subsequent to the contemporaneous emotional state exceeding the requisite minimum state of distraction, generating an alert in the computer data processing system indicating a readiness of the child patient to receive the selected specific medical procedure.

2. The method of claim 1, further comprising, generating the table by:
receiving in memory of the computer data processing system from over a computer communications network, biometric data pertaining to different children in different geographic locations, and also diversion data for each of the different children engaging in different activities;
computationally correlating the biometric data with the diversion data and the different activities in a table in the memory so as to record a cause-and-effect relationship between each one of the activities and a degree of diversion observed in the different children; and,
storing the table in fixed storage of the computer data processing system.

3. The method of claim 1, wherein the alert is a text message directed for transmission to a mobile device of a health care practitioner assigned to the health care practitioner encounter.

4. The method of claim 1, wherein the table includes different manually supplied entries correlating a degree of diversion observed in respectively different ones of the different children and corresponding ones of the different activities.

5. The method of claim 1, wherein the contemporaneous emotional state is determined from the processing of the image in addition to sensed biometric data provided to the computer data processing system from a wearable device coupled to the child patient.

6. The method of claim 1, wherein the identified activity is the playback in the computer data processing system of particular media content.

7. A computer data processing system adapted for cognitive diversion of a child patient during medical treatment, the system comprising:
a host computing system including one or more computers, each with memory and at least one processor and at least one of the computers positioned in a treatment room of a health care facility and including a display and an imaging device;
fixed storage coupled to the host computing system;
a table stored in the fixed storage correlating different activities with different degrees of diversion; and,
a cognitive diversion module comprising program instructions executing in the memory of the host computing system, the program instructions performing:
receiving in the memory from the imaging device an image of a child patient during a health care practitioner encounter in the treatment room and processing the image to determine a contemporaneous emotional state of the child patient;
selecting in the memory a specific medical procedure and retrieving from the memory, a requisite minimum state of distraction necessary for the selected specific medical procedure;
comparing the contemporaneous emotional state to the requisite minimum state of distraction; and,
on condition that the contemporaneous emotional state is determined in the comparison to lack the requisite minimum state of distraction, identifying in the table an activity correlated with a degree of diversion that exceeds the requisite minimum state of distraction, presenting the identified activity in a display of the computer data processing system, processing additional imagery of the child patient while the child patient engages in the identified activity in order to re-determine the contemporaneous emotional state and, subsequent to the contemporaneous emotional state exceeding the requisite minimum state of distraction, generating an alert indicating a readiness of the child patient to receive the selected specific medical procedure.

8. The system of claim 7, wherein the table is generated by:
receiving in the memory from over a computer communications network, biometric data pertaining to different children in different geographic locations, and also diversion data for each of the different children engaging in different activities;
computationally correlating the biometric data with the diversion data and the different activities in a table in the memory so as to record a cause-and-effect relationship between each one of the activities and a degree of diversion observed in the different children; and,
storing the table in the fixed storage.

9. The system of claim 7, wherein the alert is a text message directed for transmission to a mobile device of a health care practitioner assigned to the health care practitioner encounter.

10. The system of claim 7, wherein the table includes different manually supplied entries correlating a degree of diversion observed in respectively different ones of the different children and corresponding ones of the different activities.

11. The system of claim 7, wherein the contemporaneous emotional state is determined from the processing of the image in addition to sensed biometric data received from a wearable device coupled to the child patient.

12. The system of claim 7, wherein the identified activity is the playback in the display of particular media content.

13. A computer program product for cognitive diversion of a child patient during medical treatment, the computer program product comprising a computer readable storage medium having program instructions embodied therewith, wherein the computer readable storage medium is not a transitory signal per se, the program instructions executable by a device to cause the device to perform a method comprising:
receiving in memory of a computer data processing system, an image of a child patient during a health care practitioner encounter and processing the image to determine a contemporaneous emotional state;
selecting in the memory of the computer data processing system a specific medical procedure and retrieving from the memory, a requisite minimum state of distraction necessary for the selected specific medical procedure;
comparing the contemporaneous emotional state to the requisite minimum state of distraction; and,
on condition that the contemporaneous emotional state is determined in the comparison to lack the requisite minimum state of distraction, identifying in a table an activity correlated with a degree of diversion that exceeds the requisite minimum state of distraction, presenting the identified activity in a display of the computer data processing system, processing additional imagery of the child patient while the child patient engages in the identified activity in order to re-determine the contemporaneous emotional state and, subsequent to the contemporaneous emotional state exceeding the requisite minimum state of distraction, generating an alert in the computer data processing system indicating a readiness of the child patient to receive the selected specific medical procedure.

14. The computer program product of claim 13, further comprising, generating the table by:
receiving in memory of the computer data processing system from over a computer communications network, biometric data pertaining to different children in different geographic locations, and also diversion data for each of the different children engaging in different activities;
computationally correlating the biometric data with the diversion data and the different activities in a table in the memory so as to record a cause-and-effect relationship between each one of the activities and a degree of diversion observed in the different children; and,
storing the table in fixed storage of the computer data processing system.

15. The computer program product of claim 13, wherein the alert is a text message directed for transmission to a mobile device of a health care practitioner assigned to the health care practitioner encounter.

16. The computer program product of claim 13, wherein the table includes different manually supplied entries correlating a degree of diversion observed in respectively different ones of the different children and corresponding ones of the different activities.

17. The computer program product of claim 13, wherein the contemporaneous emotional state is determined from the processing of the image in addition to sensed biometric data provided to the computer data processing system from a wearable device coupled to the child patient.

18. The computer program product of claim 13, wherein the identified activity is the playback in the computer data processing system of particular media content.
